(19) United States
(12) Patent Application Publication
(10) Pub. No.: US 2026/0198942 A1
(43) Pub. Date: Jul. 16, 2026

(54) THROMBECTOMY CATHETER WITH FLUID JETS

(71) Applicant: Boston Scientific Scimed, Inc., Maple Grove, MN (US)

(72) Inventors: Alyssa Madej, Minnetonka, MN (US); David Brajkovic, Minneapolis, MN (US)

(73) Assignee: Boston Scientific Scimed, Inc., Maple Grove, MN (US)

(21) Appl. No.: 19/446,020

(22) Filed: Jan. 12, 2026

Related U.S. Application Data
(60) Provisional application No. 63/744,591, filed on Jan. 13, 2025.

(51) Int. Cl. A61B 17/22; A61B 17/00; A61B 17/3203

(52) U.S. Cl. CPC A61B 17/22; A61B 17/3203; A61B 2017/00199; A61B 2017/00398; A61B 2017/00539; A61B 2017/00778; A61B 2017/22082

(57) ABSTRACT

Aspiration catheters and systems are disclosed including a thrombectomy catheter which includes a catheter body extending from a proximal end region to a distal end region and includes a catheter lumen extending between the proximal end region and the distal end region. A high-pressure fluid supply tube may extend through the catheter lumen from the catheter body proximal end region toward the catheter body distal end region. The high-pressure fluid supply tube having a longitudinal axis and adapted for communication with a fluid source near the catheter body proximal end region and including a plurality of angled jet orifices with an elliptical cross-section oriented perpendicular to the longitudinal axis of the high-pressure fluid supply tube.

### CROSS-REFERENCE TO RELATED APPLICATION

This application claims the benefit of U.S. Provisional Patent Application Ser. No. 63/744,591, filed Jan. 13, 2025, entitled “THROMBECTOMY CATHETER WITH FLUID JETS”, which is incorporated by reference herein in its entirety.

### TECHNICAL FIELD

The present disclosure pertains to aspiration catheters and related devices, systems and procedures. More particularly, the present disclosure pertains to aspiration catheters, devices and systems suitable for thrombectomy and similar treatments.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### BRIEF SUMMARY

This disclosure provides design, material, manufacturing method, and use alternatives for medical devices, including aspiration catheters, thrombectomy catheters and associated devices and systems.

In a first example, a thrombectomy catheter may include a catheter body extending from a proximal end region to a distal end region and include a catheter lumen extending between the proximal end region and the distal end region. In this and other examples, a high-pressure fluid supply tube may extend through the catheter lumen from the catheter body proximal end region toward the catheter body distal end region, the high-pressure fluid supply tube having a longitudinal axis and configured and/or otherwise adapted for communication with a fluid source near the catheter body proximal end region. The high-pressure fluid supply tube of this and other examples may also include a plurality of angled jet orifices for expelling a fluid jet from the high-pressure fluid supply tube within the catheter lumen in an angled direction non-perpendicular to the longitudinal axis, whereby at least one of the plurality of angled jet orifices have an elliptical cross-section in which a long axis of the elliptical cross-section is oriented perpendicular to the longitudinal axis of the high-pressure fluid supply tube.

Alternatively or additionally to any of the examples above, the one or more of the plurality of angled jet orifices may be angled with respect to the longitudinal axis of the high-pressure fluid supply tube at an angle of between 5 and 30 degrees.

Alternatively or additionally to any of the examples above, the one or more of the plurality of angled jet orifices may include a plurality of proximally projecting jet orifices that are angled in a proximal direction to expel a fluid jet in the proximal direction.

Alternatively or additionally to any of the examples above, the one or more of the plurality of angled jet orifices may include a distally projecting jet orifice that is angled in a distal direction to expel a fluid jet in the distal direction.

Alternatively or additionally to any of the examples above, the distally projecting jet orifice may be the distalmost one of the plurality of angled jet orifices.

Alternatively or additionally to any of the examples above, the elliptical cross-section may have an area of 1.5×10⁻⁶ inches² to 1.3×10⁻⁵ inches² (9.7×10⁻⁴ millimeters² to 8.4×10⁻³ millimeters²).

Alternatively or additionally to any of the examples above, one or more of the expelled fluid jets are expelled in a spray with a breadth exceeding an arc of 20 degrees.

Alternatively or additionally to any of the examples above, one or more of the expelled fluid jets may be expelled in a concentrated spray with a max breadth not exceeding an arc of 20 degrees.

In another non-limiting example, a thrombectomy catheter may include a catheter body extending from a proximal end region to a distal end region and include a catheter lumen extending between the proximal end region and the distal end region. A high-pressure fluid supply tube may extend through the catheter lumen from the catheter body proximal end region toward the catheter body distal end region, the high-pressure fluid supply tube having a longitudinal axis and configured for communication with a fluid source near the catheter body proximal end region. A plurality of angled jet orifices may be included for expelling a fluid jet from the high-pressure fluid supply tube within the catheter lumen in an angled direction non-perpendicular to the longitudinal axis. One or more of the plurality of angled jet orifices may be angled with respect to the longitudinal axis of the high-pressure fluid supply tube at an angle of between 5 and 30 degrees. The one or more of the plurality of angled jet orifices may have an elliptical cross-section in which a long axis of the elliptical cross-section is oriented perpendicular to the longitudinal axis of the high-pressure fluid supply tube.

Alternatively or additionally to any of the examples above, the one or more of the plurality of angled jet orifices may include a plurality of proximally projecting jet orifices that are angled in a proximal direction to expel a fluid jet in the proximal direction.

Alternatively or additionally to any of the examples above, the one or more of the plurality of angled jet orifices may include a distally projecting jet orifice that may be angled in a distal direction to expel a fluid jet in the distal direction.

Alternatively or additionally to any of the examples above, the elliptical cross-section may have an area of 1.5×10⁻⁶ inches² to 1.3×10⁻⁵ inches² (9.7×10⁻⁴ millimeters² to 8.4×10⁻³ millimeters²).

Alternatively or additionally to any of the examples above, one or more of the expelled fluid jets may be expelled in a spray with a breadth exceeding an arc of 20 degrees.

In other non-limiting examples, a thrombectomy system may include a control console in communication with a pump and a fluid source, a catheter in communication with the pump and the fluid source, whereby the catheter may include a catheter body extending from a proximal end region to a distal end region and include a catheter lumen extending between the proximal end region and the distal end region. A high-pressure fluid supply tube may extend through the catheter lumen from the catheter body proximal end region toward the catheter body distal end region. The high-pressure fluid supply tube may have a longitudinal axis and may be configured and/or otherwise adapted for communication with a fluid source near the catheter body proximal end region. A plurality of angled jet orifices may be included for expelling a fluid jet from the high-pressure fluid supply tube within the catheter lumen in an angled direction non-perpendicular to the longitudinal axis. One or more of the plurality of angled jet orifices may be angled with respect to the longitudinal axis of the high-pressure fluid supply tube at an angle of between 5 and 30 degrees. One or more of the plurality of angled jet orifices may have an elliptical cross-section in which a long axis of the elliptical cross-section may be oriented perpendicular to the longitudinal axis of the high-pressure fluid supply tube. One or more of the plurality of jet orifices may include a distally projecting jet orifice that is angled in a distal direction to expel a fluid jet in the distal direction.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an illustrative thrombectomy system;

FIG. 2 is a partially exploded perspective view of the pump, a bubble trap, a connection manifold assembly, and an associated fixture of the pump/catheter assembly for use in the thrombectomy system of FIG. 1;

FIG. 3 is a partially exploded side view of the pump, the bubble trap, the connection manifold assembly, and associated fixture of the pump/catheter assembly for use in the thrombectomy system of FIG. 1;

FIG. 4 is a longitudinal cross-sectional view of a distal end region of an illustrative thrombectomy catheter;

FIG. 5 is a top view of an outer wall of a high-pressure fluid supply tube in accordance with at least one example of the present disclosure;

FIG. 6 is a side view of a longitudinal cross-section of a high-pressure fluid supply tube in accordance with at least one example of the present disclosure;

FIG. 7 is an illustrative plot of jet spray angle versus jet orifice attributes in accordance with at least one example of the present disclosure; and

FIG. 8 is an illustrative plot of subacute clot removal versus jet orifice geometry in accordance with at least one example of the present disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

Examples of the disclosure include systems, devices, and procedures for utilizing medical devices for thrombectomy and other like treatments. Reference will now be made in detail to aspects of the disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

The following detailed description should be read with reference to the drawings in which similar structures in different drawings are numbered the same. The drawings, which are not necessarily to scale, depict illustrative embodiments and are not intended to limit the scope of the disclosure.

Thrombectomy catheters and systems may be used to remove thrombus, plaques, lesions, clots, etc. from veins or arteries. Some thrombectomy catheters may use a jet tube (i.e., high-pressure fluid supply tube) that is configured in a way that the jets point directly backward (i.e., proximally into the catheter (e.g., parallel to the shaft walls) to prevent shaft damage. However, this jet tube design may block a significant portion of the cross-sectional area of the aspiration lumen, which may in turn, decrease aspiration rates. Further, this type of jet orientation may require a side window or port which may limit the vessel diameter that the device is able to reach due to the risk of the vessel wall being pulled into the catheter side window or port as well as increasing the presence of hemolysis in the target vessels. Other jet aspiration catheters may utilize high velocity saline jets in a series to entrain fluid or clot material into and through the shaft of the catheter. While proximally facing jets may macerate any clot the jetted fluid may come into contact with and prevent clogging along a length of the catheter shaft, the distal tip of the catheter may still become clogged. The distal tip of the catheter may be at greater risk of clogging during the treatment of subacute or chronic clots. Disclosed herein are a variety of high-pressure supply tubes provided with a thrombectomy catheter which greatly reduce or eliminate the chances of clogging at the distal tip of the catheter and help to remove the clot more quickly, efficiently, and effectively.

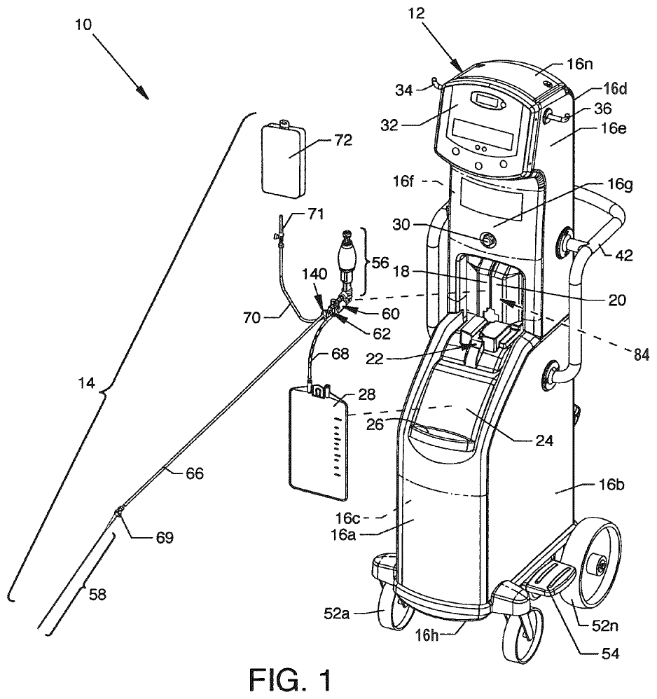

FIG. 1 is a perspective view of an illustrative thrombectomy system 10. The thrombectomy system 10 may include a control console or drive unit 12 and a pump/catheter assembly 14. In some instances, the pump/catheter assembly 14 may be a single use device in which a new pump/catheter assembly 14 may be used with the drive unit 12 for each medical procedure. Shown on the drive unit 12 are a plurality of removable panels 16a-16n about and along the drive unit 12 enclosing the internal structure of the drive unit 12. An illustrative drive unit 12 is described in commonly assigned U.S. Pat. No. 7,935,077, titled THROMBECTOMY CATHETER DEPLOYMENT SYSTEM, the disclosure of which is hereby incorporated by reference. Centrally located in the drive unit 12 and aligned to the lower region of the panel 16g may be automatically opening doors 18 and 20 which open to expose the interior of the drive unit 12 to provide access to a carriage assembly 22. The carriage assembly 22, which may accommodate components of the pump/catheter assembly 14, as discussed further herein, is shown accessible via opening the closed doors 18 and 20. The drive unit 12 may include a catch basin for collecting fluid leakage from the components of the pump/catheter assembly 14. For example, a removable drip tray 24 is shown located on the front of the drive unit 12 extending from below the carriage assembly 22 toward the panel 16a. Other configurations of catch basins are also contemplated. The drip tray 24 and a removable receptacle 26 may collectively support and accommodate an effluent collection bag, such as effluent collection bag 28 of the pump/catheter assembly 14. In other instances, the drive unit 12 may include a different structure, such as a hook for hanging the effluent collection bag 28 from, or a shelf for setting the effluent collection bag 28 on. In instances where the carriage assembly 22 is movable, a carriage assembly activation switch 30 may be provided with the drive unit 12, such as located on panel 16g, to selectively position the carriage assembly 22 inwardly or outwardly. A user interface 32, including memory capabilities, may be provided with the drive unit 12, such as located at the upper region of the drive unit 12 between the upper regions of the upper side panels 16e and 16f. Saline bag hooks 34 and 36 may extend through the panels 16e and 16f to hang saline bags therefrom. The drive unit 12 may include a handle 42 as well as a plurality of wheels 52a-52n and brake pedals 54 for wheel lockage to assist in maneuvering the drive unit 12 by medical personnel.

The pump/catheter assembly 14, which may be a disposable single-use device, is shown unattached from the drive unit 12. The pump/catheter assembly 14 includes a pump 56 and a thrombectomy catheter 58. During use, a portion of the pump/catheter assembly 14 may be secured within a portion of the drive unit 12. Other components included in the pump/catheter assembly 14 may include a bubble trap 60 attached to the pump 56, a connection manifold assembly 62 connected to the bubble trap 60, an effluent return tube 66 connected between the connection manifold assembly 62 and the thrombectomy catheter 58, a high-pressure fluid supply tube 64 attached between the output of the pump 56 and the thrombectomy catheter 58 which may be coaxially arranged inside the effluent return tube 66, a transition fixture 69 between the distal end of the effluent return tube 66 and the proximal end of the thrombectomy catheter 58, an effluent waste tube 68 connecting the effluent collection bag 28 to the connection manifold assembly 62, and a fluid supply tube 70 having a bag spike 71 connecting a fluid supply bag 72 (e.g., a saline bag) to the connection manifold assembly 62. The fluid supply tube 70 may be in fluid communication with the interior of the bubble trap 60 to provide fluid from the fluid supply bag 72 to the pump 56 and then to the thrombectomy catheter 58 through the high-pressure fluid supply tube 64.

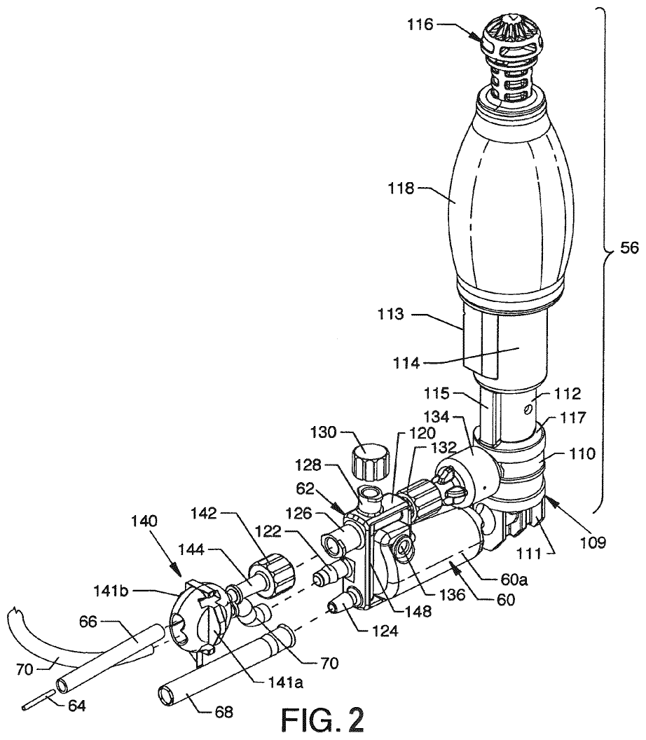

FIG. 2 is a partially exploded perspective view of several components of the pump/catheter assembly 14 generally including the pump 56, the bubble trap 60, the connection manifold assembly 62, and a fixture 140. The pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 to contain the pump 56 within the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example. A data plate 113 may also be included on the top body 114 for the inclusion of a barcode, an RFID (radio frequency identification) tag, or other informational displays to determine operational parameters of the device.

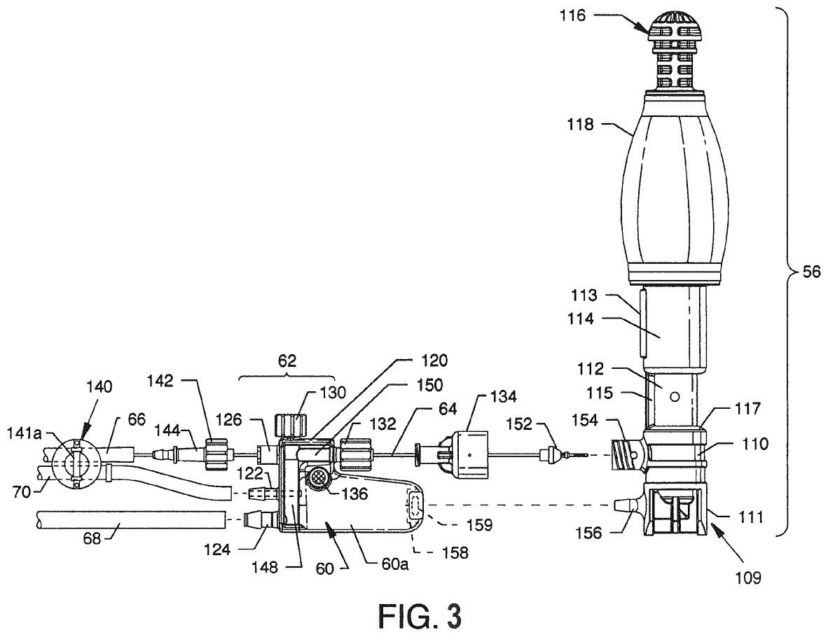

The pump 56 may include a hemispherically-shaped pump piston head 116 having a flexible boot 118 connected to and extending between the top body 114 and the pump piston head 116. In some instances, the geometrically configured lower portion 111 of the base 109 may serve as a mount for one end of the bubble trap 60 (FIG. 3).

The connection manifold assembly 62 may be secured directly to the other end of the bubble trap 60 and in some instances may include a bracket 120 to which is attached to a vertically oriented tubular manifold 148 having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 connectingly extending between the connection manifold assembly 62 and the upper portion 110 of the base 109.

The bubble trap 60 may include mating halves of which one mating half 60a is shown. A hydrophobic filter 136 may be included at the upper forward region of the bubble trap half 60a. Another hydrophobic filter may be included on the second bubble trap half (not explicitly shown) which opposes the hydrophobic filter 136 on the bubble trap half 60a.

The fixture 140, and components associated therewith, assists in support and connection of the effluent return tube 66 to the effluent return port 126 by a connector 142 combined continuously with a connection tube 144, and also assists in support, passage and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting a roller pump (not explicitly shown) provided with the drive unit 12, such as located in the carriage assembly 22 or adjacent thereto.

FIG. 3 is a partially exploded side view of the elements of FIG. 2 illustrating the relationship of the pump 56, the bubble trap 60, the connection manifold assembly 62, and the fixture 140. Also shown is the vertically oriented tubular manifold 148 secured to the bracket 120. The effluent outlet port 124 may be connected to and in fluid communication with the lower interior of the tubular manifold 148. The effluent return port 126 may be connected to and in fluid communication with the upper interior of the tubular manifold 148. Also connecting to the tubular manifold 148 is a horizontally aligned passage port 150 and associated connector 132, each opposing the effluent return port 126. The passage port 150 may accommodate the high-pressure fluid supply tube 64 which extends distally through the lumen (not explicitly shown) of the passage port 150, the connector 132, the upper region of the tubular manifold 148, the effluent return port 126, the connector 142, the connection tube 144, and into and through the effluent return tube 66 to connect to the thrombectomy catheter 58 (FIG. 1). The proximal end of the high-pressure fluid supply tube 64 includes a high-pressure fitting 152 located near the proximal end of the high-pressure fluid supply tube 64 to facilitate connection of the high-pressure fluid supply tube 64 in fluid communication with the interior of the pump 56. The proximal end of the high-pressure fluid supply tube 64, which is the inlet to the high-pressure fluid supply tube 64, may include a plurality of very small holes (not shown) comprising a filter at the proximal end thereof. The connector 134, which may have internal and/or external threads, may be aligned over and about the high-pressure fluid supply tube 64 distal to the high-pressure fitting 152 and threadingly engage a threaded connection port 154 extending horizontally from the upper portion 110 of the base 109 of the pump 56. The connector 134 may be rotated to threadably engage the high-pressure fitting 152 with a corresponding mating threaded structure provided with the pump 56. A connector 132 may be utilized to engage the externally threaded end of the connector 134 to secure the connector 134, and thus the pump 56, to the connection manifold assembly 62 and to provide for fixation of the bubble trap 60 to the pump 56. In addition, direct connection and fluid communication between the pump 56 and the bubble trap 60 may be provided by a horizontally oriented pump fluid inlet port 156 which engages a corresponding receptor port 158 and seal 159 interior to one end of the bubble trap 60. The fluid inlet port 122 located on the bracket 120 may extend behind the tubular manifold 148 to communicate with the interior of the bubble trap 60 for fluid (e.g., saline) debubbling, whereby unpressurized fluid (e.g., saline) is made available for use by the pump 56.

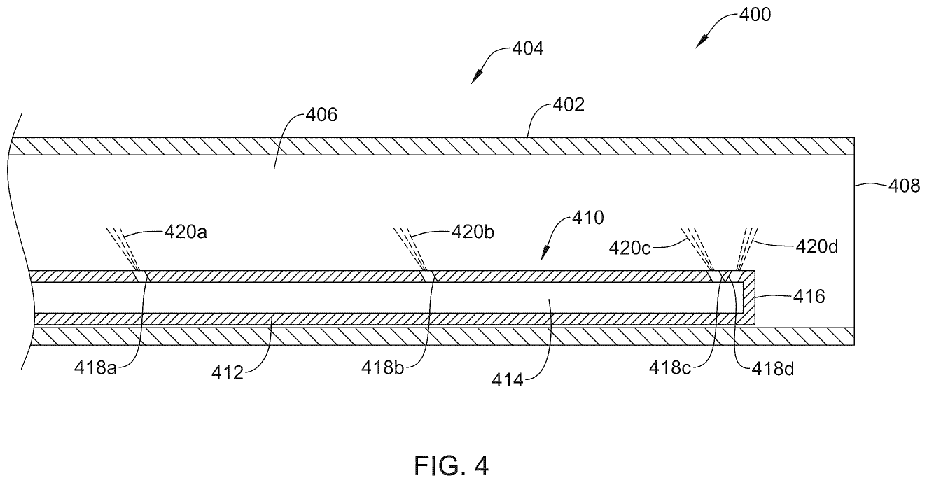

FIG. 4 is a cross-sectional view of a distal end region 404 of an illustrative thrombectomy catheter 400. The thrombectomy catheter 400 may be one illustrative example of the thrombectomy catheter 58 described above. The thrombectomy catheter 400 may include a tubular member or catheter body 402 extending from a proximal end region (not explicitly shown) configured to remain outside the body to a distal end region 404. The catheter body 402 may be one illustrative example of, or be in fluid communication with, the effluent return tube 66 of the thrombectomy catheter 58 described above. A lumen 406 may extend from the proximal end region to the distal end region 404 of the catheter body 402. The catheter body 402 may terminate at a distally facing distal opening 408 at the distal end of the catheter body 402. In some instances, the distal opening 408 may be in a plane that extends generally orthogonal to a longitudinal axis of the catheter body 402. In other instances, the distal opening 408 may be in a plane that extends generally oblique to a longitudinal axis of the catheter body 402. Generally, the distal opening 408 may be an entrainment inflow orifice. While not explicitly shown, the catheter body 402 may include one or more markers (e.g., radiopaque marker bands) disposed along the catheter body 402. Further, while not explicitly shown, in some embodiments, the catheter body 402 may include one or more openings extending through a sidewall thereof, if desired.

The thrombectomy catheter 400 may further include a high-pressure fluid supply tube 410. The high-pressure fluid supply tube 410 may be one illustrative example of, or be in fluid communication with, the high-pressure fluid supply tube 64 of the thrombectomy catheter 58 described above. It can be appreciated that the term “high-pressure fluid supply tube” may also be referred to as a high-pressure tube, high-pressure fluid tube, high-pressure supply tube, hypotube, jet tube, and/or other like terms and/or phrases. The high-pressure fluid supply tube 410 may be disposed within and extend through the lumen 406 of the catheter body 402. The high-pressure fluid supply tube 410 may include a supply tube wall 412 defining a lumen or fluid pathway 414 extending therethrough. In at least some instances, the high-pressure fluid supply tube 410 may have a closed distal end 416. Because of this, fluid may be able to pass distally through the fluid pathway 414 but does not exit the distal end. The high-pressure fluid supply tube 410 may extend along a length of the catheter body 402 with the distal end 416 located within the lumen 406 of the catheter body 402 proximal to the distal opening 408 at the distal end of the catheter body 402. A proximal end of the high-pressure fluid supply tube 410 may be in fluid communication with the pump 56 described herein, to provide high-pressure fluid to the fluid pathway 414 of the high-pressure fluid supply tube 410.

A plurality of jet orifices 418a-d (collectively, 418) may be defined along and/or within the supply tube wall 412. For example, the supply tube wall 412 may include two, three, four, five, six, or more jet orifices 418. The jet orifices 418 may be spaced along the supply tube wall 412 at any desired intervals. For example, each of the jet orifices 418 may be equidistantly spaced from adjacent jet orifices 418 along the length of the supply tube wall 412. In other instances, the jet orifices 418 may be arranged such that the spacing between adjacent jet orifices 418 near the distal end of the supply tube wall 412 is closer than the spacing between adjacent jet orifices 418 near the proximal end of the supply tube wall 412. For instance, the spacing between the orifices 418 may gradually increase as you move proximally along the length of the shaft, or the spacing may increase in a step-wise configuration. In some instances, some or all of the jet orifices 418 may be axially aligned along the supply tube wall 412. In other instances, one or more of the jet orifices 418 may be circumferentially offset from one another about the supply tube wall 412. A number of patterns are contemplated including a helical pattern, a sinusoidal pattern, a curvilinear pattern, a circumferential pattern, a pattern where no two jet orifices 418 are disposed at the same axial location, a regular pattern including two or more jet orifices 418 disposed at the same axial location, an irregular pattern (where some of the jet orifices 418 may or may not be disposed at the same axial location), an array of orifices (i.e., a circumferential and/or linear and/or curvilinear arrangement of multiple orifices), and/or any combination or permutation of the aforementioned and/or the like.

The jet orifices 418 may be formed using a suitable method such as electron discharge machining, etching, cutting (e.g., including laser cutting), laser machining, programmed laser machining, laser boring (i.e., the boring of orifices and/or holes through the application of laser technology), programmed laser boring, 3-D printing, or the like. In some instances, one or more of the jet orifices 418 may have a substantially round shape. In other instances, one or more of the jet orifices 418 may have a substantially non-round shape. In some instances, jet orifices 418 may have an elliptical shape, a substantially elliptical shape, an elliptical cross-section, a substantially elliptical cross-section, an oval shape, a substantially oval shape, an oval cross-section, a substantially oval cross-section, an ovular shape, a substantially ovular shape, an ovular cross-section, a substantially ovular cross-section or any combination or permutation of the aforementioned or any of the like. In some instances, the jet orifices 418 may be beveled, tapered, or otherwise include a beveled and/or tapered surface and/or a beveled and/or tapered bore hole defining and/or demonstrating interior characteristics of the jet orifices 418. It is contemplated that a size and/or a shape of the jet orifices 418 may be varied to vary the velocity of the fluid exiting the jet orifices. For example, decreasing the size of the jet orifices 418 may increase the velocity of the fluid exiting the jet orifices 418. In some embodiments, the size of the jet orifices 418 may be varied based on the pressure capacity of the thrombectomy system, the number of jet orifices, the dimensions of the high-pressure fluid supply tube 410 (e.g., length, wall thickness, inner diameter, etc.), and/or combinations thereof.

In some instances, the geometry of the jet orifices 418 may allow higher velocities or maintained velocities (i.e., velocity or velocities which remain constant) after a decrease in pressure provided to the jet orifices 418 and that which is germane to the innovative jet orifice geometries, cross-sections and/or shapes disclosed herein, including but not limited to jet orifice geometries, cross-sections, and/or shapes that are elliptical, substantially elliptical, transversely elliptical (i.e., where the long or longest dimension of an elliptical or like orifice extends transversely across the circumference of the high-pressure fluid supply tube and/or where the long or longest dimension of the elliptical or like orifice extends perpendicular to a longitudinal axis of the high-pressure fluid supply tube), oval, substantially oval, transversely oval, ovular, substantially ovular, transversely ovular, or through preferential combinations and/or permutations of the aforementioned.

In some examples, the jet orifices 418, which may have an elliptical or substantially elliptical cross section, may have a cross-sectional dimension parallel to the longitudinal axis of the high-pressure fluid supply tube 410 in the range of about 0.001 inches (0.0254 mm) to about 0.003 inches (0.0762 mm), in the range of about 0.001 inches (0.0254 mm) to about 0.0025 inches (0.0635 mm), about 0.0015 inches (0.0381 mm) to about 0.0025 inches (0.0635 mm), or about 0.0018 inches (0.0457 mm) to about 0.0022 inches (0.0559 mm). However, the jet orifices 418 can have a cross-sectional dimension parallel to the longitudinal axis of the high-pressure fluid supply tube 410 of less than 0.0018 inches (0.0457 mm) or greater than 0.0022 inches (0.00559 mm), as desired.

Infusion of motive fluid through the lumen 414 of the supply tube wall 412 may result in fluid being jetted through the jet orifices 418 and the generation of a proximally directed aspiration force. At least some of the jet orifices 418a-c may be angled in a proximal direction or otherwise designed to infuse fluid (e.g., a motive fluid, a liquid, a gas or air, steam, a fluid with particles disposed therein, or the like) through the jet orifices 418a-c and into the lumen 406 of the catheter body 402 in a generally proximal direction as depicted by lines 420a-c representing motive jetted fluid projecting generally proximally from the jet orifices 418a-c. For example, each of the jet orifices 418a-c may be arranged at an acute angle to the longitudinal axis of the supply tube wall 412 such that the jet orifices 418a-c angle in a proximal direction. In some embodiments, one or more of the jet orifices 418d may be designed to infuse fluid (e.g., a motive fluid, a liquid, a gas or air, steam, a fluid with particles disposed therein, or the like) through the jet orifice(s) 418d and into the lumen 406 of the catheter body 402 in a generally distal direction as depicted by lines 420d representing motive jetted fluid projecting generally distally from the jet orifice 418d. For example, the jet orifice 418d may be arranged at an oblique angle to the longitudinal axis of the supply tube wall 412 such that the jet orifice 418d angles in a distal direction. It is contemplated that an angle of the jet orifices 418 and thus the motive jetted fluid 420 may be varied to adjust the velocity of the fluid exiting the jet orifices 418.

As further described herein, the supply tube wall 412 may include one or more, or a plurality of proximally oriented or directed jet orifices 418a, 418b, 418c (i.e., jet orifices configured to direct fluid infused through the lumen 414 of the supply tube wall 412 in a proximal direction) and the supply tube wall 412 may include one or more, or a plurality of distally oriented or directed jet orifices 418d (i.e., jet orifices configured to direct fluid infused through the lumen 414 of the supply tube wall 412 in a distal direction). In some examples, the distally projecting jet orifice 418d may be axially aligned with one or more of the proximally projecting jet orifices 418a-c. In other examples, the distally projecting jet orifice 418d may be circumferentially offset from one or more of the proximally projecting jet orifices 418a-c. For example, the distally projecting jet orifice 418d may be circumferentially offset from one or more of the proximally projecting jet orifices 418a-c by in the range of about 10° to about 350° or about 45° to about 135°.

The distally projecting jet orifice 418d may be the distalmost jet orifice, with the proximally projecting jet orifices 418a-c positioned proximal of the distally projecting jet orifice 418d. However, this is not required. In some embodiments, the distally projecting jet orifice 418d may be positioned proximal to at least one proximally projecting jet orifice 418a-c. While the supply tube wall 412 is illustrated as including only a single distally projecting jet orifice 418d, the supply tube wall 412 may include more than one distally projecting jet orifice, as desired. When more than one distally projecting jet orifice 418d is provided, the distally projecting jet orifices may be positioned at differing axial and/or circumferential locations from one another or similar axial and/or circumferential locations as one another, as desired. The distally projecting jet orifice(s) 418d may break up particles as they are drawn into the lumen 406 of the catheter body 402 while the proximally projecting jet orifices 418a-c may move particles proximally along the catheter body 402.

The performance of the thrombectomy catheter 400 and the high-pressure fluid supply tube 410 may be directly related to the velocity of the motive jetted fluid 420 exiting the jet orifices 418 and the shear-induced turbulent flux created by the jetted motive fluid 420. For example, the more powerful the jetted motive fluid 420, the higher the aspiration rates may be. It is further contemplated that the performance of the jet-powered aspiration catheter 400 may be directly related to the speed at which the clot can be entrained into the catheter 400, macerated, and removed from the body. Any clogging that occurs within the catheter body 402 may reduce or completely stop the removal of the clot. The addition of the distally projecting jet orifice 418d may macerate any clot that enters the distal opening 408 of the catheter body 402 thus helping prevent clogging. For example, at the point of impingement of the distally oriented motive jetted fluid 420d the motive jetted fluid 420d may deflect distally creating flow out the tip of the distal opening 408 of the catheter body 402, effectively macerating any clot that enters the tip of the device, and eliminating or reducing risk of the distal opening 408 of the catheter body 402 becoming blocked or clogged. It is contemplated that the properties (size, shape, angle, number, spacing, etc.) of the jet orifices 418 may be varied to obtain a fluid velocity that creates an optimum de-clogging effect without hindering the proximal flow of a clot within the lumen 406 of the catheter body 402 or the clot evacuation rate.

The distally projecting jet orifice 418d may be proximally spaced a distance from the distal opening 408 of the catheter body 402. It is contemplated that the longitudinal location of the distally projecting jet orifice 418d on the supply tube wall 412 and relative to the distal opening 408 of the catheter body 402 may be varied based on a size of the aperture of the distally projecting jet orifice 418d, the velocity of the fluid within the lumen 414 of the supply tube wall 412, the angle of the distally projecting jet orifice 418d, or combinations thereof, etc. to ensure the distally oriented motive jetted fluid 420d impinges the inner surface of the catheter body 402. In one illustrative example, the distally projecting jet orifice 418d may be positioned such that the distally oriented motive jetted fluid 420d impinges an inner surface of the catheter body 402 such that the distally oriented motive jetted fluid 420d does not damage the vessel. For example, the distally projecting jet orifice 418d may be positioned such that the distally oriented motive jetted fluid 420d impinges an inner surface of the catheter body 402 in the range of about 0.070 inches (1.778 millimeters) to about 0.090 inches (2.286 millimeters) proximal to the distal end of the catheter body 402. This is just one example. The impingement location of the motive jetted fluid 420d of the distally projecting jet orifice 418d may be less than 0.070 inches (1.778 millimeters) or more than 0.090 inches (2.286 millimeters) proximal to the distal end of the catheter body 402, as desired.

In some instances, the jet orifices 418 may be oriented at an angle relative to the longitudinal axis of the supply tube wall 412. For example, the proximally projecting jet orifices 418a-c may be oriented at an oblique (e.g., acute) angle relative to the longitudinal axis of the supply tube wall 412 and/or oriented at an angle greater than zero degrees and less than ninety degrees relative to the longitudinal axis of the supply tube wall 412. It is contemplated that a distally projecting jet orifice 418d may be oriented at an oblique (e.g., obtuse) angle relative to the longitudinal axis of the supply tube wall 412 and/or oriented at an angle greater than 90 degrees and less than 180 degrees relative to the longitudinal axis of the supply tube wall 412. In other instances, the jet orifices 418 may be oriented perpendicular to the longitudinal axis of the supply tube wall 412 (e.g., at an angle of about 90 degrees relative to the longitudinal axis of the supply tube wall 412). The angle may or may not be the same for all the jet orifices 418.

In at least some instances, the jet orifices 418 may be understood as being arranged in series. In other words, the jet orifices 418 may be arranged such that adjacent jet orifices 418 are spaced longitudinally apart at various locations along the longitudinal axis of the supply tube wall 412. For example, the jet orifices 418 may be uniformly or non-uniformly spaced along a length and/or other dimensions of the supply tube wall 412. This may position the jet orifices 418 at axially and/or circumferentially spaced apart locations within the catheter body 402 and along the length thereof. For example, the jet orifices 418 may be spaced along an entire length of the supply tube wall 412 and correspondingly along an entire length of the catheter body 402, or portions thereof, as desired. In some examples, the jet orifices 418 may be spaced at intervals in the range of every 5 inches (12.7 centimeters (cm)) to every 15 inches (38.1 cm), or in the range of every 6 inches (15.2 cm) to every 12 inches (30.5 cm) along a length of the supply tube wall 412. In other instances, the spacing between the jet orifices 418 may be less than every 5 inches (12.7 cm) or greater than every 15 inches (38.1 cm). Accordingly, motive fluid leaves via the jet orifices 418 forming a jetted motive fluid 420a-d (collectively, 420). In some instances, the jetted motive fluid 420 may reach speeds of 17,150 centimeters/second or greater (e.g., half the speed of sound, or greater). This jetted motive fluid 420 enters an entrainment material where the shear layer between the two causes turbulence, mixing, and transfer of momentum. Entrainment material may enter the distal opening 408 and then may be urged proximally by momentum transfer. As the mixture of jetted motive fluid 420 and entrainment material migrates proximally, the material may sequentially approach a number of jet orifices 418. Upon interaction with the jetted motive fluid 420 from each individual jet orifice 418, the momentum in the entrainment material mixture may increase, and the thrombogenic material may more readily flow proximally through the catheter body 402 for removal. The increase in momentum may allow for the catheter body 402 to be used without a second or outflow orifice (e.g., positioned proximally of the distal opening 408). Alternatively, some of the entrapped thrombogenic material may exit the catheter body 402 through a second orifice (not shown), e.g., in a sidewall of the catheter body 402, positioned proximal to the distal opening 408, recirculate to the distal opening 408 (e.g., one or more times), and then move proximally through the lumen 406 of the catheter body 402.

It is further contemplated that the distally oriented motive jetted fluid 420d may be partially to fully entrained by the force generated by the proximally oriented motive jetted fluid 420a-c. When the clot/thrombus reaches the distally oriented motive jetted fluid 420d, the shear stress may masticate the clot/thrombus. It is contemplated that when the distal opening 408 of the catheter body 402 is sealed with a clot/thrombus, the force generated by the proximally oriented motive jetted fluid 420a-c may be transferred to the surface of the clot/thrombus in a proximal direction. As a result, the distally oriented motive jetted fluid 420d may no longer be entrained and may transfer force in the distal direction to the surface of the clot/thrombus. Thus, when the distal opening 408 is clogged or plugged, an extreme shear mechanism of action is created where the distal and proximal force vectors combine together to focus all of the shear stress to the surface of the clot/thrombus to masticate the clot/thrombus and unplug the distal opening 408. It is contemplated that the shear stress on the clot/thrombus may be much larger in magnitude when the distally oriented motive jetted fluid 420d is at a smaller angle (e.g., closer to 180 degrees relative to the longitudinal axis of the supply tube wall 412 than to orthogonal to the longitudinal axis of the supply tube wall 412).

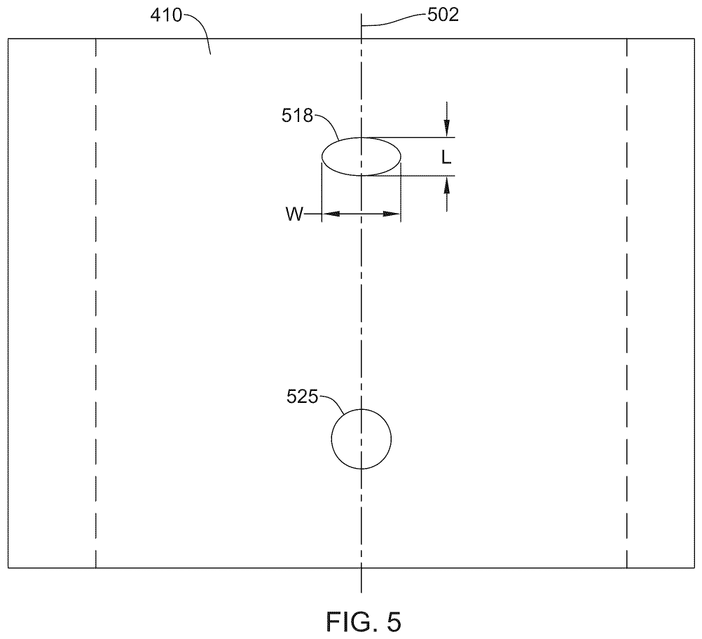

Turning to FIG. 5, an example longitudinal section of the exemplary high-pressure fluid supply tube 410 is shown. The high-pressure fluid supply tube 410 has a longitudinal axis 502 that runs along the length of high-pressure fluid supply tube 410. In this and other examples, the longitudinal axis 502 may serve as a reference point for measuring angles with respect to the longitudinal axis 502, including but not limited to, jet angle, jet spray angle, concentrated jet spray angle, orifice angle, orifice orientation angle, orifice bore hole angle, orifice taper angle, orifice bore hole taper angle, and any of the like.

The section of the high-pressure fluid supply tube 410 shown in FIG. 5 illustrates one example configuration of the jet orifices 418, described above, which may be a representative example of any one or more of the jet orifices 418a-d of the high-pressure fluid supply tube 410 described above. As shown in the example of FIG. 5 and applicable to other examples described herein, the high-pressure fluid supply tube 410 may include one or more elliptical orifices 518 (which may be a representative example of any one or more of the jet orifices 418a-d of the high-pressure fluid supply tube 410 described above). Elliptical orifices 518 and others described herein may have a maximum dimension or width W, and a minimum dimension or length L. The width W is greater than the length L. In other words, the length of the major axis (i.e., width W) is greater than the length of the minor axis (i.e., length L). The elliptical orifice 518 may be arranged with the minimum dimension or minor axis (length L) extending parallel to the longitudinal axis 502 of the high-pressure fluid supply tube 410 and the maximum dimension or major axis (width W) extending perpendicular to the longitudinal axis 502 of the high-pressure fluid supply tube 410. In some instances, the width W may be within a range of about 0.0015 inches (0.0381 mm) to about 0.009 inches (0.2286 mm) or more, in the range of about 0.002 inches (0.0508 mm) to about 0.006 inches (0.1524 mm), or in the range of about 0.006 inches (0.1524 mm) to about 0.009 inches (0.2286 mm). In this and other examples, the length L may be about 0.001 inches (0.0254 mm) or less, about 0.002 inches (0.0508 mm) or less, about 0.0025 inches (0.0635 mm) or less, or about 0.003 inches (0.0762 mm) or less. In some instances, the length L may be within a range of about 0.001 inches (0.0254 mm) to about 0.005 inches (0.127 mm), in the range of 0.001 inches (0.0254 mm) to about 0.003 inches (0.0762 mm), in the range of 0.001 inches (0.0254 mm) to about 0.0025 inches (0.0635 mm), or in the range of 0.001 inches (0.0254 mm) to about 0.002 inches (0.0508 mm), for example. However, other length and width ranges and specificities are contemplated. It is noted that the area of an ellipse is pi*(W/2)(L/2). It is contemplated that in some instances the cross-sectional area of the elliptical orifice 518 may be in the range of 1.5×10⁻⁶ inches² to 1.3×10⁻⁵ inches² (9.7×10⁻⁴ millimeters² to 8.4×10⁻³ millimeters²), in the range of 3.0×10⁻⁶ inches² to 8.0×10⁻⁶ inches² (1.9×10⁻³ millimeters² to 5.2×10⁻³ millimeters²), in the range of 3.4×10⁻⁶ inches² to 1.3×10⁻⁵ inches² (2.2×10⁻³ millimeters² to 8.4×10⁻³ millimeters²), or in the range of 3.0×10⁻⁶ inches² to 1.0×10⁻⁵ inches² (1.9×10⁻³ millimeters² to 6.5×10⁻³ millimeters²), for example. Other sizes are contemplated. Below, in Table 1, are some exemplary dimensions for the elliptical orifice 518.

TABLE 1

| major | | minor | | |
|---|---|---|---|---|
| diameter (W) | major radius | diameter (L) | minor radius | Area |
| (inches) | (inches) | (inches) | (inches) | inches\hat{ }2 |
| 0.002 | 0.001 | 0.001 | 0.0005 | 1.5708E−06 |
| 0.004 | 0.002 | 0.001 | 0.0005 | 3.14159E−06 |
| 0.006 | 0.003 | 0.001 | 0.0005 | 4.71239E−06 |
| 0.003 | 0.0015 | 0.0025 | 0.00125 | 5.89049E−06 |
| 0.004 | 0.002 | 0.0025 | 0.00125 | 7.85398E−06 |
| 0.006 | 0.003 | 0.0025 | 0.00125 | 1.1781E−05 |
| major | | minor | | |
| diameter | major radius | diameter | minor radius | Area |
| (mm) | (mm) | (mm) | (mm) | mm\hat{ }2 |
| 0.0508 | 0.0254 | 0.0254 | 0.0127 | 0.001013415 |
| 0.1016 | 0.0508 | 0.0254 | 0.0127 | 0.00202683 |
| 0.1524 | 0.0762 | 0.0254 | 0.0127 | 0.003040245 |
| 0.0762 | 0.0381 | 0.0635 | 0.03175 | 0.003800306 |
| 0.1016 | 0.0508 | 0.0635 | 0.03175 | 0.005067075 |
| 0.1524 | 0.0762 | 0.0635 | 0.03175 | 0.007600612 |

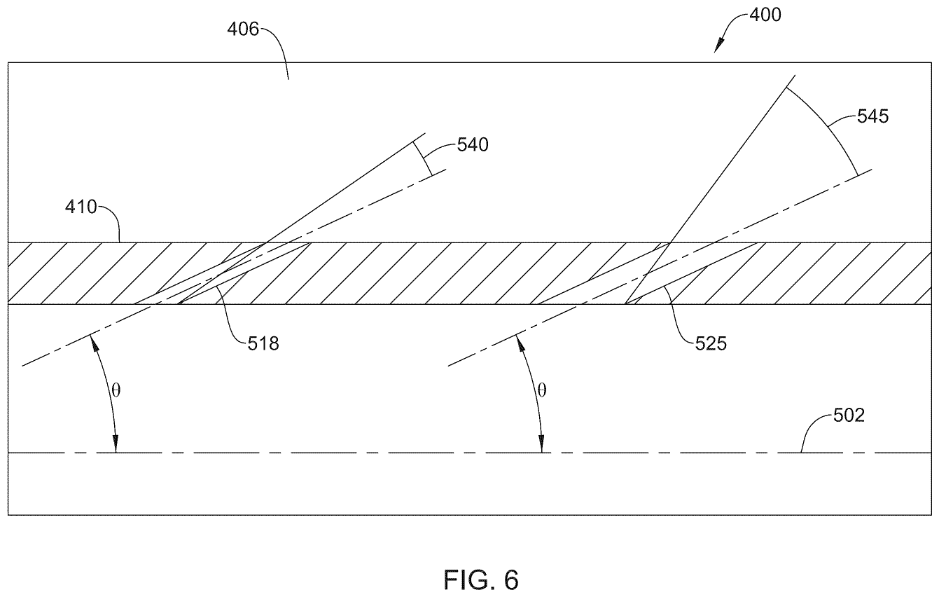

Continuing to FIG. 6, a side view of a cross-section of the example high-pressure fluid supply tube 410 and catheter 400 are shown. The high-pressure fluid supply tube 410 may be nested within the catheter body 402 of the catheter 400 and/or within one or more catheter lumens (such as catheter lumen 406) within the catheter 400. One or more elliptical orifices 518 may be drilled, cut and/or otherwise bored within one or more walls of the high-pressure fluid supply tube 410 and may allow for high-pressure fluid to be expelled at an acute jet angle such as the angle θ. The angle θ may be any desired acute angle, such as an angle in the range of 20° to 70°, in the range of 20° to 60°, in the range of 20° to 45°, or in the range of 20° to 30°, for example. In the embodiment shown in FIG. 6, the angle is chosen as 25°. Elliptical orifices 518 (and other orifices described herein) may be bored via laser drilling, a programmed laser drilling routine, laser etching techniques, programmed laser etching techniques, laser-boring techniques (i.e., utilizing a laser and/or lasers to bore a hole, orifice, bore hole, etc.), programmed laser-boring techniques, laser machining techniques, programmed laser machining techniques, and/or other like techniques. In a non-limiting sense, jet angle 540 shown in FIG. 6 may represent and/or conform to the path of least resistance for jetted fluid being expelled through elliptical orifice 518 and/or other additional jet orifices contemplated herein relative to the central axis of the elliptical orifice 518. The path of least resistance is a property inherent to fluids and a fundamental principle in fluid mechanics, in which the flow of fluid will follow the path of least resistance to the flow of fluid. It can be appreciated that this aforementioned principle may be applied and manipulated via the structure, shape and/or geometry and/or area and/or volume of the orifices described herein.

Comparatively, FIG. 6 also displays a circular orifice 525, which may yield a remarkably different jet angle 545 relative to the central axis of the circular orifice 525, which also may typically demonstrate the path of least resistance of a jetted fluid being expelled from the circular orifice 525. The presence and discussion of the circular orifice 525 is intended for comparative purposes to compare the shape, configuration, and functionality of an elliptical orifice 518 to those of a circular orifice 525. In this and other examples, no circular orifices may be present and the orifices provided may all be elliptical, substantially elliptical, oval, substantially oval, ovular and/or substantially ovular. In yet other non-limiting examples, some of the jet orifices may be elliptical, substantially elliptical, oval, substantially oval, ovular and/or substantially ovular, however any combination and/or permutation of the aforementioned is contemplated herein.

As shown in FIG. 6, the jet angle 540 of the fluid expelled from the elliptical orifice 518 relative to the central axis of the elliptical orifice 518 may be less than the jet angle 545 of the fluid expelled from the circular orifice 525 relative to the central axis of the circular orifice 525. Accordingly, the elliptical orifice 518 may reduce the angle of the path of least resistance through the elliptical orifice 518 (while having an equivalent cross-sectional area, and thus equivalent fluid pressure and speed), thereby decreasing the angle of the jet spray relative to the longitudinal axis 502 of the high-pressure fluid supply tube 410.

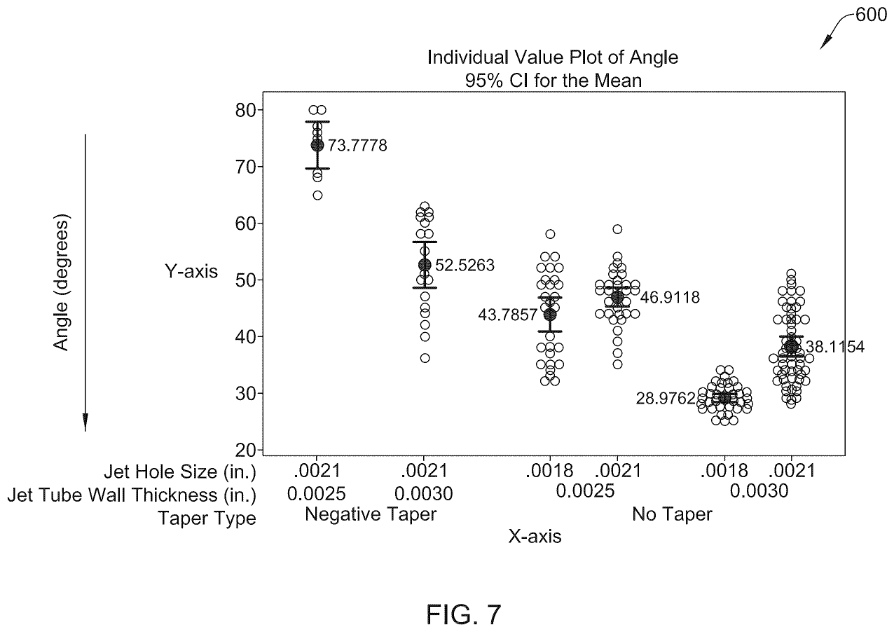

FIG. 7 illustrates a value plot of fluid jet angles obtained from varying orifices contemplated herein which may possess varying, different, complementary and/or alternative taper angles such as a negative taper (in which the jet angle and/or orifice faces proximally) and/or a positive taper (in which the jet angle and/or orifice faces distally). FIG. 7 compares jet hole sizes (i.e., diameter) of 0.0018 inches (0.0457 mm) and 0.0021 inches (0.0533 mm), and jet tube wall thickness of 0.0025 inches (0.0635 mm) and 0.0030 inches (0.0762 mm). As shown by the plot in FIG. 7, there is an observed relationship between jet hole size (in inches), jet tube (i.e., high-pressure fluid supply tube) wall thickness (in inches), the type of taper (i.e., negative, positive, etc.) shown on the X-axis and the angle of expelled jet spray (in degrees) which is tabulated and charted along the Y-axis. The mean value for the expelled jet spray angle in each configuration is shown next to the associated plotted values. As shown along the Y-axis, angles of jet spray expulsion are correlated with taper angles which include negative taper angles, and jet spray expulsion angles observed when no taper is present within and/or applied to the jet orifices described herein. As shown in FIG. 7, changing the diameter (or shape) of the jet orifice changes the path of least resistance of the fluid expelled from the jet orifice, and thus the fluid jet angle of the expelled fluid. It can also be seen that jet hole size coupled with taper angle may also yield differing jet spray angles (i.e., angles of resultant and observed jet spray expelled from the given orifices charted on the plot of FIG. 7). It can further be shown that greater taper angles may result in a greater angle of resultant jet spray expelled from the orifices under observed conditions, such as an approximately 74° angle with respect to a longitudinal axis of the high-pressure fluid supply tube observed with a jet hole size (i.e., diameter) of 0.0021 inches, (0.0533 mm) a jet tube wall thickness (i.e., a high-pressure fluid supply tube wall thickness) of 0.0025 inches (0.0635 mm) and a negative taper angle bored for the produced orifice. Table 2 below provides additional and supporting data which is plotted and illustrated in FIG. 7.

Table 2, below, displays various jet tube wall thicknesses compared with taper types, jet orifice (jet hole) sizes (i.e., diameter), and resultant jet spray directions (i.e., proximal, distal, etc.) and resultant jet spray angles. As shown and tabulated below, jet orifices with no taper and with negative taper (i.e., in which the jet angle and/or orifice faces proximally) were evaluated. Jet orifice size (i.e., diameter of jet orifice) is shown ranging in value from 0.0018 inches (0.0457 mm) to 0.0021 inches (0.0533 mm) although other dimensions are contemplated by this disclosure.

TABLE 2

| Jet | Jet Tube | | | | |
|---|---|---|---|---|---|
| Tube | Wall | | Jet Hole | | |
| Sample | Thickness | Taper | Size | Jet | Angle |
| # | (inches) | Type | (inches) | Direction | (degrees) |
| 1 | 0.0025 | No Taper | .0021 | Distal | 116 |
| 1 | 0.0025 | No Taper | .0021 | Proximal | 48 |
| 1 | 0.0025 | No Taper | .0021 | Proximal | 49 |
| 1 | 0.0025 | No Taper | .0021 | Proximal | 49 |
| 1 | 0.0025 | No Taper | .0021 | Proximal | 44 |
| 1 | 0.0025 | No Taper | .0021 | Proximal | 47 |
| 2 | 0.0025 | No Taper | .0021 | Distal | 115 |
| 2 | 0.0025 | No Taper | .0021 | Proximal | 44 |
| 2 | 0.0025 | No Taper | .0021 | Proximal | 46 |
| 2 | 0.0025 | No Taper | .0021 | Proximal | 48 |
| 2 | 0.0025 | No Taper | .0021 | Proximal | 49 |
| 2 | 0.0025 | No Taper | .0021 | Proximal | 51 |
| 3 | 0.0025 | No Taper | .0021 | Distal | 111 |
| 3 | 0.0025 | No Taper | .0021 | Proximal | 48 |
| 3 | 0.0025 | No Taper | .0021 | Proximal | 37 |
| 3 | 0.0025 | No Taper | .0021 | Proximal | 35 |
| 3 | 0.0025 | No Taper | .0021 | Proximal | 43 |
| 3 | 0.0025 | No Taper | .0021 | Proximal | 45 |
| 4 | 0.0025 | No Taper | .0021 | Distal | 115 |
| 4 | 0.0025 | No Taper | .0021 | Proximal | 43 |
| 4 | 0.0025 | No Taper | .0021 | Proximal | 44 |
| 4 | 0.0025 | No Taper | .0021 | Proximal | 44 |
| 4 | 0.0025 | No Taper | .0021 | Proximal | 41 |
| 4 | 0.0025 | No Taper | .0021 | Proximal | 39 |
| 5 | 0.0025 | No Taper | .0021 | Distal | 116 |
| 5 | 0.0025 | No Taper | .0021 | Proximal | 48 |
| 5 | 0.0025 | No Taper | .0021 | Proximal | 54 |
| 5 | 0.0025 | No Taper | .0021 | Proximal | 48 |
| 5 | 0.0025 | No Taper | .0021 | Proximal | |
| 5 | 0.0025 | No Taper | .0021 | Proximal | 59 |
| 6 | 0.0025 | No Taper | .0021 | Distal | 109 |
| 6 | 0.0025 | No Taper | .0021 | Proximal | 49 |
| 6 | 0.0025 | No Taper | .0021 | Proximal | 46 |
| 6 | 0.0025 | No Taper | .0021 | Proximal | 52 |
| 6 | 0.0025 | No Taper | .0021 | Proximal | 49 |
| 6 | 0.0025 | No Taper | .0021 | Proximal | 46 |
| 7 | 0.0025 | No Taper | .0021 | Distal | 113 |
| 7 | 0.0025 | No Taper | .0021 | Proximal | 52 |
| 7 | 0.0025 | No Taper | .0021 | Proximal | 53 |
| 7 | 0.0025 | No Taper | .0021 | Proximal | 44 |
| 7 | 0.0025 | No Taper | .0021 | Proximal | 50 |
| 7 | 0.0025 | No Taper | .0021 | Proximal | 51 |
| 8 | 0.0030 | Negative Taper | .0021 | Distal | 119 |
| 8 | 0.0030 | Negative Taper | .0021 | Proximal | 50 |
| 8 | 0.0030 | Negative Taper | .0021 | Proximal | 63 |
| 8 | 0.0030 | Negative Taper | .0021 | Proximal | 45 |
| 8 | 0.0030 | Negative Taper | .0021 | Proximal | 58 |
| 8 | 0.0030 | Negative Taper | .0021 | Proximal | 42 |
| 9 | 0.0030 | Negative Taper | .0021 | Distal | 121 |
| 9 | 0.0030 | Negative Taper | .0021 | Proximal | 62 |
| 9 | 0.0030 | Negative Taper | .0021 | Proximal | 60 |
| 9 | 0.0030 | Negative Taper | .0021 | Proximal | 61 |
| 9 | 0.0030 | Negative Taper | .0021 | Proximal | 62 |
| 10 | 0.0030 | Negative Taper | .0021 | Distal | 118 |
| 10 | 0.0030 | Negative Taper | .0021 | Proximal | 40 |
| 10 | 0.0030 | Negative Taper | .0021 | Proximal | 53 |
| 10 | 0.0030 | Negative Taper | .0021 | Proximal | 58 |
| 10 | 0.0030 | Negative Taper | .0021 | Proximal | 44 |
| 10 | 0.0030 | Negative Taper | .0021 | Proximal | 61 |
| 11 | 0.0030 | Negative Taper | .0021 | Distal | 120 |
| 11 | 0.0030 | Negative Taper | .0021 | Proximal | 55 |
| 11 | 0.0030 | Negative Taper | .0021 | Proximal | 50 |
| 11 | 0.0030 | Negative Taper | .0021 | Proximal | 51 |
| 11 | 0.0030 | Negative Taper | .0021 | Proximal | 36 |
| 11 | 0.0030 | Negative Taper | .0021 | Proximal | 47 |
| 12 | 0.0025 | Negative Taper | .0021 | Distal | 105 |
| 12 | 0.0025 | Negative Taper | .0021 | Proximal | 77 |
| 12 | 0.0025 | Negative Taper | .0021 | Proximal | 76 |
| 12 | 0.0025 | Negative Taper | .0021 | Proximal | 80 |
| 12 | 0.0025 | Negative Taper | .0021 | Proximal | 80 |
| 13 | 0.0025 | Negative Taper | .0021 | Distal | 112 |
| 13 | 0.0025 | Negative Taper | .0021 | Proximal | 69 |
| 13 | 0.0025 | Negative Taper | .0021 | Proximal | 65 |
| 13 | 0.0025 | Negative Taper | .0021 | Proximal | 75 |
| 13 | 0.0025 | Negative Taper | .0021 | Proximal | 74 |
| 13 | 0.0025 | Negative Taper | .0021 | Proximal | 68 |
| 14 | 0.0030 | No Taper | .0021 | Distal | 118 |
| 14 | 0.0030 | No Taper | .0021 | Proximal | 42 |
| 14 | 0.0030 | No Taper | .0021 | Proximal | 32 |
| 14 | 0.0030 | No Taper | .0021 | Proximal | 35 |
| 14 | 0.0030 | No Taper | .0021 | Proximal | 33 |
| 14 | 0.0030 | No Taper | .0021 | Proximal | 30 |
| 15 | 0.0030 | No Taper | .0021 | Distal | 117 |
| 15 | 0.0030 | No Taper | .0021 | Proximal | 36 |
| 15 | 0.0030 | No Taper | .0021 | Proximal | 38 |
| 15 | 0.0030 | No Taper | .0021 | Proximal | 39 |
| 15 | 0.0030 | No Taper | .0021 | Proximal | 35 |
| 15 | 0.0030 | No Taper | .0021 | Proximal | 34 |
| 16 | 0.0030 | No Taper | .0021 | Proximal | 49 |
| 16 | 0.0030 | No Taper | .0021 | Proximal | 40 |
| 16 | 0.0030 | No Taper | .0021 | Proximal | 51 |
| 16 | 0.0030 | No Taper | .0021 | Proximal | 48 |
| 17 | 0.0030 | No Taper | .0021 | Distal | 119 |
| 17 | 0.0030 | No Taper | .0021 | Proximal | 48 |
| 17 | 0.0030 | No Taper | .0021 | Proximal | 44 |
| 17 | 0.0030 | No Taper | .0021 | Proximal | 41 |
| 17 | 0.0030 | No Taper | .0021 | Proximal | 50 |
| 18 | 0.0025 | No Taper | .0018 | Distal | 119 |
| 18 | 0.0025 | No Taper | .0018 | Proximal | 50 |
| 18 | 0.0025 | No Taper | .0018 | Proximal | 38 |
| 18 | 0.0025 | No Taper | .0018 | Proximal | 38 |
| 18 | 0.0025 | No Taper | .0018 | Proximal | 33 |
| 18 | 0.0025 | No Taper | .0018 | Proximal | 32 |
| 19 | 0.0025 | No Taper | .0018 | Distal | 119 |
| 19 | 0.0025 | No Taper | .0018 | Proximal | 38 |
| 19 | 0.0025 | No Taper | .0018 | Proximal | 35 |
| 19 | 0.0025 | No Taper | .0018 | Proximal | 37 |
| 19 | 0.0025 | No Taper | .0018 | Proximal | 35 |
| 19 | 0.0025 | No Taper | .0018 | Proximal | 34 |
| 19 | 0.0025 | No Taper | .0018 | Proximal | 32 |
| 20 | 0.0030 | No Taper | .0021 | Proximal | 45 |
| 20 | 0.0030 | No Taper | .0021 | Proximal | 48 |
| 20 | 0.0030 | No Taper | .0021 | Proximal | 39 |
| 21 | 0.0030 | No Taper | .0021 | Proximal | 36 |
| 21 | 0.0030 | No Taper | .0021 | Proximal | 43 |
| 21 | 0.0030 | No Taper | .0021 | Proximal | 43 |
| 21 | 0.0030 | No Taper | .0021 | Proximal | 31 |
| 21 | 0.0030 | No Taper | .0021 | Proximal | 32 |
| 21 | 0.0030 | No Taper | .0021 | Proximal | 45 |
| 22 | 0.0030 | No Taper | .0021 | Proximal | 46 |
| 22 | 0.0030 | No Taper | .0021 | Proximal | 43 |
| 22 | 0.0030 | No Taper | .0021 | Proximal | 46 |
| 22 | 0.0030 | No Taper | .0021 | Proximal | 46 |
| 22 | 0.0030 | No Taper | .0021 | Proximal | 43 |
| 23 | 0.0030 | No Taper | .0018 | Proximal | 25 |
| 23 | 0.0030 | No Taper | .0018 | Proximal | 30 |
| 24 | 0.0030 | No Taper | .0018 | Proximal | 28 |
| 24 | 0.0030 | No Taper | .0018 | Proximal | 27 |
| 24 | 0.0030 | No Taper | .0018 | Proximal | 33 |
| 24 | 0.0030 | No Taper | .0018 | Proximal | 30 |
| 24 | 0.0030 | No Taper | .0018 | Proximal | 27 |
| 24 | 0.0030 | No Taper | .0018 | Proximal | 31 |
| 24 | 0.0030 | No Taper | .0018 | Proximal | 30 |
| 25 | 0.0030 | No Taper | .0018 | Proximal | 28 |
| 25 | 0.0030 | No Taper | .0018 | Proximal | 28 |
| 25 | 0.0030 | No Taper | .0018 | Proximal | 27 |
| 25 | 0.0030 | No Taper | .0018 | Proximal | 29 |
| 25 | 0.0030 | No Taper | .0018 | Proximal | 25 |
| 26 | 0.0030 | No Taper | .0018 | Proximal | 29 |
| 26 | 0.0030 | No Taper | .0018 | Proximal | 26 |
| 26 | 0.0030 | No Taper | .0018 | Proximal | 28 |
| 26 | 0.0030 | No Taper | .0018 | Proximal | 32 |
| 27 | 0.0030 | No Taper | .0018 | Proximal | 29 |
| 27 | 0.0030 | No Taper | .0018 | Proximal | 29 |
| 27 | 0.0030 | No Taper | .0018 | Proximal | 28 |
| 27 | 0.0030 | No Taper | .0018 | Proximal | 28 |
| 27 | 0.0030 | No Taper | .0018 | Proximal | 27 |
| 27 | 0.0030 | No Taper | .0018 | Proximal | 29 |
| 28 | 0.0030 | No Taper | .0018 | Proximal | 28 |
| 28 | 0.0030 | No Taper | .0018 | Proximal | 30 |
| 28 | 0.0030 | No Taper | .0018 | Proximal | 27 |
| 28 | 0.0030 | No Taper | .0018 | Proximal | 31 |
| 28 | 0.0030 | No Taper | .0018 | Proximal | 32 |
| 29 | 0.0030 | No Taper | .0018 | Proximal | 26 |
| 29 | 0.0030 | No Taper | .0018 | Proximal | 27 |
| 30 | 0.0030 | No Taper | .0018 | Proximal | 31 |
| 30 | 0.0030 | No Taper | .0018 | Proximal | 30 |
| 30 | 0.0030 | No Taper | .0018 | Proximal | 29 |
| 30 | 0.0030 | No Taper | .0018 | Proximal | 32 |
| 30 | 0.0030 | No Taper | .0018 | Proximal | 34 |
| 31 | 0.0030 | No Taper | .0018 | Proximal | 30 |
| 31 | 0.0030 | No Taper | .0018 | Proximal | 28 |
| 31 | 0.0030 | No Taper | .0018 | Proximal | 25 |
| 32 | 0.0030 | No Taper | .0018 | Proximal | 31 |
| 32 | 0.0030 | No Taper | .0018 | Proximal | 34 |
| 32 | 0.0030 | No Taper | .0018 | Proximal | 29 |
| 33 | 0.0030 | No Taper | .0021 | Proximal | 36 |
| 33 | 0.0030 | No Taper | .0021 | Proximal | 38 |
| 33 | 0.0030 | No Taper | .0021 | Proximal | 37 |
| 33 | 0.0030 | No Taper | .0021 | Proximal | 37 |
| 34 | 0.0030 | No Taper | .0021 | Proximal | 37 |
| 34 | 0.0030 | No Taper | .0021 | Proximal | 32 |
| 34 | 0.0030 | No Taper | .0021 | Proximal | 32 |
| 34 | 0.0030 | No Taper | .0021 | Proximal | 34 |
| 35 | 0.0030 | No Taper | .0021 | Proximal | 36 |
| 36 | 0.0030 | No Taper | .0021 | Proximal | 35 |
| 36 | 0.0030 | No Taper | .0021 | Proximal | 33 |
| 36 | 0.0030 | No Taper | .0021 | Proximal | 33 |
| 37 | 0.0030 | No Taper | .0021 | Proximal | 34 |
| 37 | 0.0030 | No Taper | .0021 | Proximal | 34 |
| 38 | 0.0030 | No Taper | .0021 | Proximal | 31 |
| 38 | 0.0030 | No Taper | .0021 | Proximal | 29 |
| 39 | 0.0030 | No Taper | .0021 | Proximal | 36 |
| 39 | 0.0030 | No Taper | .0021 | Proximal | 30 |
| 40 | 0.0030 | No Taper | .0021 | Proximal | 29 |
| 40 | 0.0030 | No Taper | .0021 | Proximal | 28 |
| 41 | 0.0025 | No Taper | .0018 | Proximal | 45 |
| 41 | 0.0025 | No Taper | .0018 | Proximal | 47 |
| 42 | 0.0025 | No Taper | .0018 | Proximal | 49 |
| 42 | 0.0025 | No Taper | .0018 | Proximal | 50 |
| 42 | 0.0025 | No Taper | .0018 | Proximal | 46 |
| 43 | 0.0025 | No Taper | .0018 | Proximal | 52 |
| 43 | 0.0025 | No Taper | .0018 | Proximal | 40 |
| 44 | 0.0025 | No Taper | .0018 | Proximal | 52 |
| 44 | 0.0025 | No Taper | .0018 | Proximal | 54 |
| 45 | 0.0025 | No Taper | .0018 | Proximal | 49 |
| 45 | 0.0025 | No Taper | .0018 | Proximal | 54 |
| 45 | 0.0025 | No Taper | .0018 | Proximal | 58 |
| 45 | 0.0025 | No Taper | .0018 | Proximal | 35 |
| 46 | 0.0025 | No Taper | .0018 | Proximal | 52 |
| 46 | 0.0025 | No Taper | .0018 | Proximal | 49 |
| 46 | 0.0025 | No Taper | .0018 | Proximal | 47 |
| 46 | 0.0025 | No Taper | .0018 | Proximal | 45 |

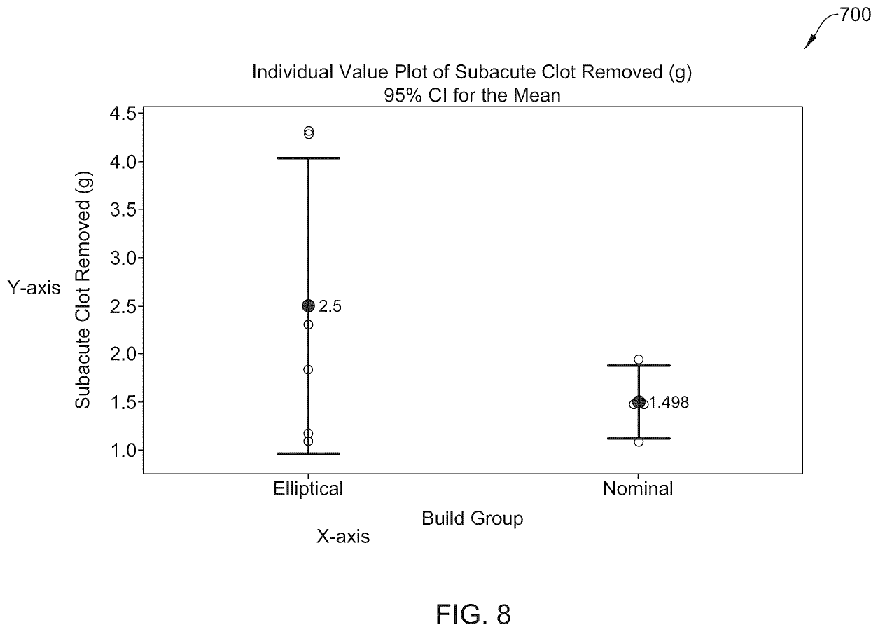

Returning to the drawings, FIG. 8 illustrates a value plot of subacute clot removed in relation to elliptical and nominal (i.e., circular) shaped orifices. As shown in FIG. 8 and applicable to other examples disclosed herein, it can be seen that elliptical orifices are capable of removing more subacute clot in many instances in comparison to the utilization of nominal orifices such as those that are circular. Further shown by the relationship between elliptical jet hole geometry and the amount of subacute clot removed, it can be seen that an elliptical jet hole geometry provides a greater range of outcomes in removal of subacute clot. In other words, it can be appreciated that the devices and systems disclosed herein may provide a wide range of clot removal options and treatments given the specific geometry (i.e., area, surface area, taper angle, orifice angle, etc.) of an elliptical orifice such that a proper and/or optimal treatment may be tailored to specific and/or myriad treatment environments. For example, in certain instances a great amount of subacute clot may require removal and may be accomplished with the elliptical orifices in combination with the disclosed features recited herein. Further, and in other non-limiting instances, a lesser amount of subacute clot may require removal for various reasons, including but not limited to the nature of the treatment site, the health and/or age of the patient or subject, and other reasons and/or forecasts affecting the recommended course of treatment for using the devices and systems disclosed herein. It can be seen that although certain elliptical orifices may remove lesser amounts of subacute clot (which may be an aim of a physician and/or practitioner), the amount of subacute clot removed is still typically greater and more effective than procedures relying upon circular orifices within a high-pressure fluid supply tube as borne by the results plotted in FIG. 8.

Table 3 below depicts results of clot removal comparison between a thrombectomy catheter having elliptical jet holes and a thrombectomy catheter having nominal (i.e., circular) jet holes. Tests were run with the pump of the thrombectomy system having a down-stroke velocity (“dsv”) and fluid pressure (“kpsi”) as provided in Table 3. A subtraction of post-test clot weight (in grams) from pre-test clot weight (g) reveals macerated clot removed (MAB) in grams after controlling for pressure and waste products and is provided below. Total waste collected (“Waste”) was also recorded and represents the total volume collected in the waste bag, including clot material, water from the test bath, and the high pressure jet fluid.

TABLE 3

| | | | | Pre- | Post- | | | | |
|---|---|---|---|---|---|---|---|---|---|
| | Jet | Distal Jet | | Test | Test | | | | |
| | Tube | Hole | | Clot | Clot | MAB Clot | | | |
| Build | Sample | Placement | Net Evac | Weight | Weight | Removed | Waste | | |
| Group | # | (in) | (mL/min) | (g) | (g) | (g) | (mL) | kpsi | dsv |
| Elliptical | A | 0.036 | 73 | 53.14 | 48.85 | 4.29 | 251 | 15.23 | 5 |
| Elliptical | B | 0.032 | 80.5 | 52.66 | 48.34 | 4.32 | 153.5 | 14.78 | 5 |
| Elliptical | C | 0.037 | 69 | 53.58 | 52.49 | 1.09 | 123 | 13.89 | 6.5 |
| Elliptical | D | 0.035 | 82 | 53.57 | 51.27 | 2.3 | 140 | 15.32 | 6.5 |
| Elliptical | E | 0.031 | 89 | 50.45 | 49.28 | 1.17 | 104 | 14.14 | 6 |
| Elliptical | F | 0.033 | 77 | 50.43 | 48.6 | 1.83 | 107 | 14.8 | 6 |
| Nominal | G | 0.03 | 141 | 49.99 | 48.48 | 1.51 | 112 | 15.2 | 5 |
| Nominal | H | * | 116 | 54.13 | 52.65 | 1.48 | 98 | 14.78 | 5 |
| Nominal | I | * | 98 | 50.29 | 48.35 | 1.94 | 232.3 | 15.12 | 5.5 |
| Nominal | J | 0.032 | 55 | 50.52 | 49.04 | 1.48 | | 14.89 | 5.5 |
| Nominal | K | 0.031 | 57 | 50.64 | 49.56 | 1.08 | 105.5 | 15.17 | 5 |

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A thrombectomy catheter, comprising:
a catheter body extending from a proximal end region to a distal end region and including a catheter lumen extending between the proximal end region and the distal end region;
a high-pressure fluid supply tube extending through the catheter lumen from the catheter body proximal end region toward the catheter body distal end region, the high-pressure fluid supply tube comprising a longitudinal axis and configured for communication with a fluid source near the catheter body proximal end region;
a plurality of angled jet orifices for expelling a fluid jet from the high-pressure fluid supply tube within the catheter lumen in an angled direction non-perpendicular to the longitudinal axis;
wherein one or more of the plurality of angled jet orifices have an elliptical cross-section in which a long axis of the elliptical cross-section is oriented perpendicular to the longitudinal axis of the high-pressure fluid supply tube.

2. The thrombectomy catheter of claim 1, wherein the one or more of the plurality of angled jet orifices are angled with respect to the longitudinal axis of the high-pressure fluid supply tube at an angle of between 5 and 30 degrees.

3. The thrombectomy catheter of claim 1, wherein the one or more of the plurality of angled jet orifices include a plurality of proximally projecting jet orifices that are angled in a proximal direction to expel a fluid jet in the proximal direction.

4. The thrombectomy catheter of claim 1, wherein the one or more of the plurality of angled jet orifices includes a distally projecting jet orifice that is angled in a distal direction to expel a fluid jet in the distal direction.

5. The thrombectomy catheter of claim 4, wherein the distally projecting jet orifice is the distalmost one of the plurality of angled jet orifices.

6. The thrombectomy catheter of claim 4, wherein the distally projecting jet orifice is angled with respect to the longitudinal axis of the high-pressure fluid supply tube at an angle of between 5 and 30 degrees.

7. The thrombectomy catheter of claim 1, wherein the elliptical cross-section has an area of 1.5×10⁻⁶ inches² to 1.3×10⁻⁵ inches² (9.7×10⁻⁴ millimeters² to 8.4×10⁻³ millimeters²).

8. The thrombectomy catheter of claim 1, wherein one or more of the expelled fluid jets are expelled in a spray with a breadth exceeding an arc of 20 degrees.

9. The thrombectomy catheter of claim 1, wherein one or more of the expelled fluid jets are expelled in a concentrated spray with a maximum breadth not exceeding an arc of 20 degrees.

10. A thrombectomy catheter, comprising:
a catheter body extending from a proximal end region to a distal end region and including a catheter lumen extending between the proximal end region and the distal end region;
a high-pressure fluid supply tube extending through the catheter lumen from the catheter body proximal end region toward the catheter body distal end region, the high-pressure fluid supply tube comprising a longitudinal axis and configured for communication with a fluid source near the catheter body proximal end region;
a plurality of angled jet orifices for expelling a fluid jet from the high-pressure fluid supply tube within the catheter lumen in an angled direction non-perpendicular to the longitudinal axis;
wherein one or more of the plurality of angled jet orifices are angled with respect to the longitudinal axis of the high-pressure fluid supply tube at an angle of between 5 and 30 degrees;
wherein the one or more of the plurality of angled jet orifices have an elliptical cross-section in which a long axis of the elliptical cross-section is oriented perpendicular to the longitudinal axis of the high-pressure fluid supply tube.

11. The thrombectomy catheter of claim 10, wherein the one or more of the plurality of angled jet orifices include a plurality of proximally projecting jet orifices that are angled in a proximal direction to expel a fluid jet in the proximal direction.

12. The thrombectomy catheter of claim 10, wherein the one or more of the plurality of angled jet orifices includes a distally projecting jet orifice that is angled in a distal direction to expel a fluid jet in the distal direction.

13. The thrombectomy catheter of claim 12, wherein the distally projecting jet orifice is the distalmost one of the plurality of angled jet orifices.

14. The thrombectomy catheter of claim 12, wherein the distally projecting jet orifice is angled with respect to the longitudinal axis of the high-pressure fluid supply tube at an angle of between 5 and 30 degrees.

15. The thrombectomy catheter of claim 10, wherein the elliptical cross-section has an area of 1.5×10⁻⁶ inches² to 1.3×10⁻⁵ inches² (9.7×10⁻⁴ millimeters² to 8.4×10⁻³ millimeters²).

16. The thrombectomy catheter of claim 10, wherein one or more of the expelled fluid jets are expelled in a spray with a breadth exceeding an arc of 20 degrees.

17. The thrombectomy catheter of claim 10, wherein one or more of the expelled fluid jets are expelled in a concentrated spray with a maximum breadth not exceeding an arc of 20 degrees.

18. A thrombectomy system, comprising:
a control console in communication with a pump and a fluid source;
a catheter in communication with the pump and the fluid source;
wherein the catheter comprises a catheter body extending from a proximal end region to a distal end region and including a catheter lumen extending between the proximal end region and the distal end region;
a high-pressure fluid supply tube extending through the catheter lumen from the catheter body proximal end region toward the catheter body distal end region, the high-pressure fluid supply tube comprising a longitudinal axis and configured for communication with a fluid source near the catheter body proximal end region;
a plurality of angled jet orifices for expelling a fluid jet from the high-pressure fluid supply tube within the catheter lumen in an angled direction non-perpendicular to the longitudinal axis;
wherein one or more of the plurality of angled jet orifices are angled with respect to the longitudinal axis of the high-pressure fluid supply tube at an angle of between 5 and 30 degrees;
wherein the one or more of the angled jet orifices have an elliptical cross-section in which a long axis of the elliptical cross-section is oriented perpendicular to the longitudinal axis of the high-pressure fluid supply tube; and
wherein one or more of the plurality of angled jet orifices includes a distally projecting jet orifice that is angled in a distal direction to expel a fluid jet in the distal direction.

19. The thrombectomy system of claim 18, wherein one or more of the expelled fluid jets are expelled in a spray with a breadth exceeding an arc of 20 degrees.

20. The thrombectomy system of claim 18, wherein one or more of the expelled fluid jets are expelled in a concentrated spray with a maximum breadth not exceeding an arc of 20 degrees.
